(19) United States
(12) Patent Application Publication
(10) Pub. No.: US 2006/0002904 A9
(43) Pub. Date: Jan. 5, 2006

(54) Receptor and related products and methods

(76) Inventor: Byoung S. Kwon, Eulju-kun (KR)

Correspondence Address: BARNES & THORNBURG, 11 SOUTH MERIDIAN, INDIANAPOLIS, IN, 46204, US

(21) Appl. No.: 10/170,997

(22) Filed: Jun. 12, 2002

Related U.S. Application Data
(63) Continuation of application No. 08/955,573, filed on Oct. 22, 1997, now abandoned.
Application No. 08/955,573 is a continuation of application No. 08/460,976, filed on Jun. 5, 1995, now abandoned.
Application No. 08/460,976 is a division of application No. 08/122,796, filed on Sep. 16, 1993, now abandoned.
Application No. 08/122,796 is a continuation-in-part of application No. 08/012,269, filed on Feb. 1, 1993, now Pat. No. 6,362,325.

(51) Int. Cl. A61K 48/00; C07H 21/04; C12P 21/02; C12N 5/06

(52) U.S. Cl. 424/93.21; 435/69.1; 435/320.1; 435/325; 530/350; 536/23.5

(57) ABSTRACT

The human receptor H4-1BB has been isolated, sequenced and disclosed herein. The cDNA of the human receptor H4-1BB is about 65% homologous to the mouse cDNA 4-1BB and was isolated by using probes derived from cDNA 4-1BB. A fusion protein for detecting cell membrane ligands to human receptor protein H4-1BB was developed. It comprises the extracellular portion of the receptor protein H4-1BB and a detection protein (alkaline phosphatase) bound to the portion of the receptor protein H4-1BB. B-cells that have expressed a ligand to receptor protein H4-1BB can be treated with cells that have expressed receptor protein H4-1BB and B-cell proliferation may be induced. The use of H4-1BB to block H4-1BB ligand binding has practical application in the suppression of the immune system during organ transplantation. A monoclonal antibody against H4-1BB can be used to enhance T-cell proliferation by treating T-cells that have expressed receptor protein H4-1BB with the anti H4-1BB monoclonal antibody. Tumors transfected with H4-1BBL may be capable of delivering antigen-specific signals as well as the co-stimulatory signals and can be killed by human cytotoxic T lymphocytes.

This application is a continuation-in-part of co-pending application Ser. No. 08/012,269 filed Feb. 1, 1993, which is a continuation-in-part of co-pending application Ser. No. 07/922,996 filed Jul. 30, 1992, which is a continuation-in-part of copending application Ser. No. 07/267,577 filed Nov. 7, 1988.

The subject matter described herein was in part a subject invention of NIH Grants Nos. IR23AI23058-03, RO1 AI28175 and P60 KD20542 of which the present inventor was the Principal Investigator and either the Donald Guthrie Foundation for Medical Research Inc. of Guthrie Square, Sayre, Pa. 18849-1669 or Indiana University School of Medicine of Indianapolis, Ind. 46202, was the Grantee.

### FIELD OF THE PRESENT INVENTION

The present invention relates to a previously unknown human receptor protein, H4-1BB, which was isolated and identified based upon work with a homologous murine (mouse) receptor protein, 4-1BB, which was isolated and identified by specific expression of the T cell genes by the present inventor.

### BACKGROUND OF THE PRESENT INVENTION

The immune system of humans and other species requires that white blood cells be made in the bone marrow, which white blood cells include phagocytes, lymphocytes and B cells. As presently understood, the phagocytes include macrophage cells which scavenge unwanted materials such as virus protein from the system. The lymphocytes include helper T cells and killer T cells and B cells as well as other cells, including those categorized as suppressor T cells. The B cells produce the antibodies. The killer T cells physically pierce the cell and the helper T cells facilitate the whole process. In any event, the immune process is facilitated by lymphokines.

Lymphokines are the proteins by which the immune cells communicate with each other. Scientists produce them in sufficient quantities for therapeutic use against immunologic diseases. There are many known lymphokine proteins and they include the interferons, interleukin-1,2,3,4,5,6,7, colony-stimulating factors, lymphotoxin, tumor necrosis factor and erythropoietin, as well as others.

Interleukin 1, secreted from macrophages activate the helper T cells and raise the body temperature causing fever which enhances the activity of the immune cells. The activated helper T Cells produce Interleukin 2 and Interleukin 2 stimulates the helper and killer T cells to grow and divide. The helper T cells also produce another lymphokine, B cell growth factor (BCGF), which causes B cells to multiply. As the number of B cells increases, the helper T cells produce another lymphokine known as the B cell differentiating factor (BCDF), which instructs some of the B cells to stop replicating and start producing antibodies. T cells also produce a lymphokine, gamma interferon (IF), which has multiple effects like Interleukin 2. Interferon helps activate killer T cells, enabling them to attack the invading organisms. Like BCGF, interferon increases the ability of the B cells to produce antibodies. Interferon also affects the macrophages to keep them at the site of the infection and help the macrophages to digest the cells they have engulfed. Gathering momentum with each kind of lymphokine signal between the macrophages and the T cells, the lymphokines amplify the immune system response and the virus protein or other foreign matter on the infected cells is overwhelmed. There are many other lymphokines, maybe a hundred or more, which participate in the immune process. Many lymphokines are known and many are not.

Lymphokines are sometimes called intercellular peptide signals. Among scientists there is widespread use of cloned cell lines as lymphokine producers and the isolation of lymphokine mRNA has become a common technique. The mouse receptor protein, 4-1BB, was isolated and identified based on specific expression of the T cell genes using a technique identified by the present inventor in a publication (Proc. Natl. Acad. Sci. USA. 84, 2896-2900, May 1987, Immunology). The protocol reported in this publication can be used by scientists to detect virtually all of the lymphokines. The method is designed to detect virtually all mRNA expressed differentially and the mRNA sequences of the immune cells are expressed differentially (as they relate to the T cells and the killer T cells) even though the level of expression is low and the quantity of the secreted lymphokine protein is low. The present inventor believes that the analysis described in the above identified publication can reveal biologically important molecules such as lymphokines because there are many indications that biologically important or active molecules are coded by the most scarce messages. An example is a transforming growth factor (TGF) which is present as only one of a million clones.

Most T cell factors have been classically identified by recognizing biologic activities in assays, purifying the protein information. An alternative approach is to isolate putative T cell genes based upon specific expression and then demonstrate the function of the unknown molecule. Using the aforesaid modified differential screening procedure, the present inventor cloned a series of T cell subset-specific cDNAs from cloned helper T (HTL) L2 and cloned cytolytic T lymphocyte (CTL) L3.

A series of T-cell subset-specific cDNAs were isolated from cloned murine T-cells by employing a modified differential screening procedure. The nucleotide sequence and expression properties of some of the cDNA species have been reported. One of the genes not previously characterized, that encodes mouse receptor protein 4-1BB,, was studied further. These studies have led to the isolation of the human homologue to 4-1BB, H4-1BB.

### SUMMARY OF THE PRESENT INVENTION

The present invention includes the human receptor protein H4-1BB and the cDNA gene encoding for human receptor protein H4-1BB. The nucleotide sequence of the isolated cDNA is disclosed herein along with the deduced amino acid sequence. The cDNA gene identified as pH 4-1BB was deposited at the Agricultural Research Service Culture Collection and assigned the accession number: NRRL B21131

The cDNA, and fragments and derivatives thereof, can be used as a probe to isolate DNA sequences encoding for proteins similar to the receptor protein encoded by the cDNA. The cDNA of the human receptor H4-1BB is about 65% homologous to the mouse cDNA 4-1BB and was isolated by using probes derived from cDNA 4-1BB. The cDNA gene identified as p4-1BB was deposited at the American Type Culture Collection at 12301 Parklawn Drive, Rockville, Md. 20852 under ATCC No. 67825.

The human receptor protein H4-1BB can be produced by: 1) inserting the cDNA of H4-1BB into an appropriate expression vector, 2) transfecting the expression vector into an appropriate transfection host, c) growing the transfected hosts in appropriate culture media and d) purifying the receptor protein from the culture media. The protein and fragments and derivatives can be used: 1) as a probe to isolate ligands to human receptor protein H4-1BB, 2) to stimulate proliferation of B-cells expressing H4-1BB ligands, or 3) to block H4-1BB ligand binding.

B-cell proliferation can be induced by treating B-cells that have expressed a ligand to receptor protein H4-1BB with cells that have expressed receptor protein H4-1BB.

The use of H4-1BB to block H4-1BB ligand binding has practical application in the suppression of the immune system during organ transplantation. A similar costimulatory immune system pathway is being analyzed for this type of application. See “Mounting a Targeted Strike on Unwanted Immune Responses”, Jon Cohen, Science, Vol. 257, 8-7-92; “Long Term Survival of Xenogeneic Pancreatic Islet Grafts Induced by CTLA41g”, Lenschow et al, Science Vol. 257, 7-8-92; and “Immunosuppresion in Vivo by a Soluble Form of the CTLA-4 T Cell Activation Molecule”, Linsley et al, Science Vol. 257 7-8-92.

A monoclonal antibody against H4-1BB can be used to enhance T-cell proliferation by treating T-cells that have expressed receptor protein H4-1BB with the anti H4-1BB monoclonal antibody. Some tumors are potentially immunogenic but do not stimulate an effective anti-immune response in vivo. Tumors may be capable of delivering antigen-specific signals to T cells, but may not deliver the co-stimulatory signals necessary for full activation of T cells. Expression of the co-stimulatory ligand B7 on of melanoma cells was found to induce the rejection of a murine melanoma in vivo. (“Tumor Rejection After Direct Co-Stimulation of CD8⁺ T Cells by B7-Transfected Melanoma Cells”, Sarah E. Townsend and James P. Allison, Science Vol. 259, 1-5-93.) A monoclonal antibody against H4-1BB may be capable of the same effect as it is now known to enduce T cell proliferation and activation.

A fusion protein for detecting cell membrane ligands to human receptor protein H4-1BB was developed. It comprises the extracellular portion of the receptor protein H4-1BB and a detection protein (alkaline phosphatase) bound to the portion of the receptor protein H4-1BB. The portion of the receptor protein H4-1BB binds to the cell membrane ligands and binding can be detected by relative activity assays for the detection protein. The fusion protein is placed in the presence of a cell suspected to express the receptor protein H4-1BB. Then the cell is washed of any fusion protein not bound to the cell membrane ligands. Once the washed cells are placed in the presence of a substrate for the detection protein and the relative activity of the detection protein can be measured.

The primary object of the present invention is the identification of the new human receptor, H4-11BB as identified herein by its sequence.

Another object of the present invention is to teach a fusion protein comprising the extracellular portion of H4-1BB and a detection protein.

Still another object of the present invention is to teach methods of using the cDNA H4-1BB, the receptor protein H4-1BB, the monoclonal antibody and the legand for H4-1BB.

### BRIEF DESCRIPTIONS OF THE FIGURES

FIG. 1 shows the sequence for the cDNA of mouse receptor protein 4-1BB and the regions used as PCR primers to obtain the human homologue H4-1BB.

FIGS. 2a and 2b show the nucleotide sequence and the deduced amino acid sequence of human receptor H4-1BB respectively.

FIGS. 3a and 3b illustrate the molecules involved in T-cell activation.

FIGS. 4a, 4b, and 4c illustrate a normal T-cell activation pathway.

FIGS. 5a, 5b, and 5c illustrate CTLA4-lg alone, 4-1BB/AP and CTLA4-lg together and 4-1BB/AP alone espectively being used to block steps in the T-cell ctivation pathway.

### DETAILED DESCRIPTION

In the following detailed description references are made to known procedures and studies, as well as published work of the applicant. These publications are incorported herein by reference for clarity and listed in an appendix included at the end of this detailed description.

Isolation and Characterization of Mouse Receptor 4-1BB

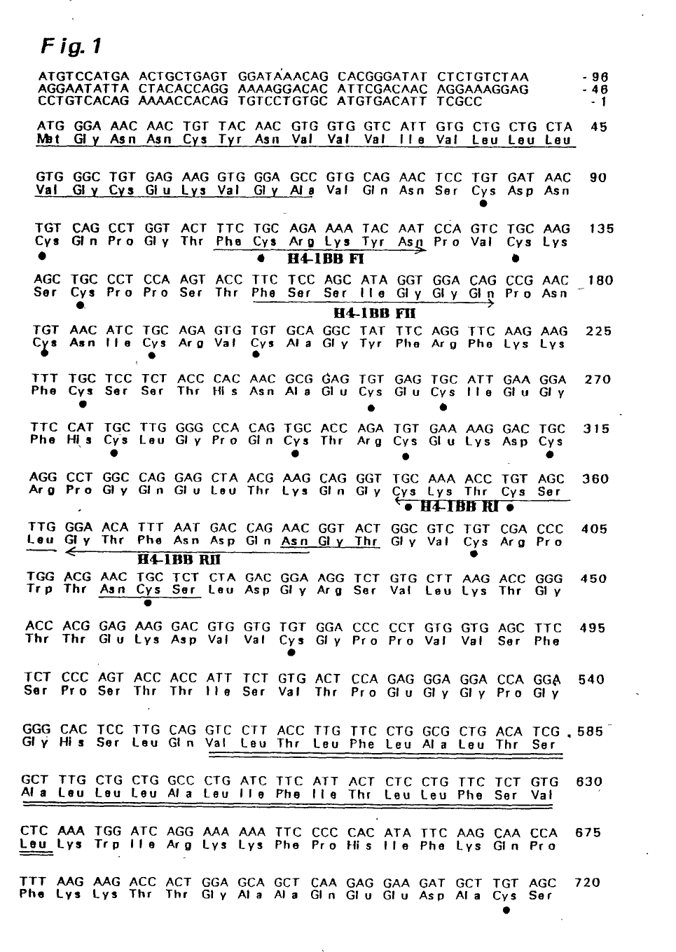

FIG. 1 shows the nucleotide sequence and the deduced amino acid sequence of the mouse receptor 4-1BB. The nucleotides of the message strand are numbered in the 5′ to 3′ direction and numbers are shown on both sides of the sequence. Nucleotide residue 1 is the A of the initiation codon ATG, and the nucleotides on the 5′ side of residue 1 are indicated by negative numbers. The predicted amino acid sequence is shown below the nucleotide sequence. Putative signal peptide is underlined. Stop codon is indicated by ( - - - ). Cysteine residues are highlighted by the dots. An unusual feature of 4-1BB sequence is that there is a potential polyadenylation signal of AATAAA at nucleotides 1158-1163 (FIG. 1 boxed). It was believed that this signal was functional because this gene produces at least two different sizes of mRNA.

The transcript of 4-1BB was inducible by concanavalin A in mouse splenocytes, T-cell clones, and hybridomas. The expression of 4-1BB transcripts was inhibited by cyclosporin A. The 4-1BB mRNA was inducible by antigen receptor stimulation but was not inducible by Il-2 stimulation in the cloned T-cells (1). The 4-1BB cDNA encodes a peptide of 256 amino acids containing a putative leader sequence, a potential membrane anchor segment, and other features of known receptor proteins. Therefore, the expression pattern of 4-1BB resembles those of lymphokine RNAs while the sequence appeared consistent with those of receptor proteins.

The major species of 4-1BB on the cell surface appears to be a 55-kDa dimer. 4-1BB also appears to exist as a 30-kDa monomer and possibly as a 110-kDa tetramer. Since these 4-1BB species were immunoprecipitated from a homogenous population of cells (T cell clone F1), all forms potentially co-exist on each cell. A comparison of peptide digests from the 4-1BB monomer and dimer will be needed to determine whether 4-1BB exists as a homodimer on the cell surface. A variety of cell surface receptors such as the insulin receptor (2), the B cell surface immunoglobulin receptor (3), the T cell Ag receptor (4), the CD28 costimulatory receptor (5), and the CD27 T cell antigen (6) are composed of disulfide-bonded subunits. Receptor dimerization may be required for ligand binding and subsequent biochemical signaling.

4-1BB is not expressed on resting T cells but is inducible by activators which deliver a complete growth stimulus to the T cell. The combination of PMA and ionomycin is capable of mimicing those signals required for T cell proliferation. Although PMA or ionomycin alone induced 4-1BB mRNA, the combination of PMA and ionomycin resulted in optimal 4-1BB expression. Furthermore, the expression of 4-1BB was not transient. When purified splenic T cells were stimulated with immobilized anti-CD3, 4-lBB mRNA was expressed and this expression was maintained for up to 96 hrs poststimulation. Cell cycle analysis will be required to confirm that 4-1BB is expressed throughout cell cycle progression.

4-1BB is structurally related to members of the nerve growth factor receptor super-family. Although these receptors possess structurally similar ligand-binding properties (cysteine-rich regions), the cytoplasmic domains of these proteins are nonconserved which could allow for diversity in transmembrane signaling. Some members of this family are involved in the T or B cell activation process. There are in vitro functional data on the OX-40, CD40 and CD27 antigens. Antibodies against the OX-40 augment the T cell response in a mixed lymphocyte reaction (7) and antibodies against CD40 enhance B-cell proliferation in the presence of a coactivator, such as PMA or CD2O antibodies, and synergize with IL-4 in vitro to induce B-cell differentiation and to generate long-term normal B cell lines (8). One inonoclonal antibody, anti-1A4, which recognizes an epitope on the CD27 molecule inhibited calcium mobilization, IL-2 secretion, helper T cell function, and T cell proliferation. On the other hand, CLB-CD27/1, another anti-CD27 mAb enhanced proliferation of hunan T cells stimulated with PHA or anti-CD3 mAb (6).

These results indicate that the CD27 molecule plays an important role in T cell activation. Except for TNFRs, NCFR and CD40, the ligands or cell surface molecules to which the members of the superfamily bind are not yet identified. Identification and characterization of the ligands to which the receptors bind will be helpful in better defining the physiologic role of 4-1BB.

To ascertain whether cell surface 4-1BB could contribute to T cell activation, the anti-4-1BB 53A2 was used as an antagonist to 4-1BB. These data suggested that 4-1BB does in fact have the potential to function as an accessory signaling molecule during T cell activation and proliferation. The addition of soluble 53A2 to purified splenic T cells stimulated with immobilized anti-CD3 resulted in an amplification of ³H thymidine incorporation compared to T cells stimulated with anti-CD3 alone. This pattern of enhancement ranged from 2- to 10-fold in three independent experiments. In the original two signal model of Bretcher and Cohn, they proposed that signal 1, the occupancy of the T cell antigen receptor (TCR), resulted in inactivation of the T cell in the absence of signal 2, which is provided by accessory cells. This has since been confirmed by a variety of studies (9). The identification of the accessory cell CD28 as a potent costimulatory receptor on T cells was a significant contribution in beginning to charactize the accessory signal(s) required for optimal T cell proliferation (10). It is possible that other cell surface molecules may contribute to these costimulatory activation requirements (11).

The biochemical signals delivered through 4-1BB are not completely known. One possibility considered was the observation that 4-1BB contains a putative p₅₆^{lck} tyrosine kinase binding domain in its cytoplasmic tail. It was later determined that p₅₆^{lck} tyrosinase kinase binds to 4-1BB. It will also be worthwhile to determine if 4-1BB-mediated signaling can regulate genes such as IL-2 and IL-2 receptor, whose expression is required for T cell activation and subsequent proliferation.

Although the precise functions of members of the Nerve Growth Factor Receptor (NGFR) family appear to be diverse, an emerging theme is one in which these molecules may contribute in various ways to a maintenance of responsiveness or viability of the particular cell type in which they are expressed. For instance, NGF is absolutely required for viability of neurons in vitro and in vivo (12). The crosslinking of CD40 by soluble antiCD40 monoclonal antibody blocks germinal center centrocytes from undergoing apoptosis in vitro (13). Signals delivered throug CD40 may also aid in maintenance of responsiveness to differentiation factors. The ligation of CD40 with anti-CD40 F(ab′)₂ fragments in the presence of IL-4 induced large increases IgE synthesis (14). Also, anti-CD40 activated naive B cells treated with IL-10 and transforming growth factor-β became committed to IgA secretion (15). In addition to sharing the molecular characteristics with the NGFR superfamily, it was noted that the 4-1BB contained a putative zinc finger structure of the yeast elF-2β protein (16). 4-1BB also shares a conserved region with the sina seven in absentia of Drosophila, which is required for correct photoreceptor cell development (17). That particular region is also similar to the protein product of the DG17 gene of Dictyostelium, whose expression is specifically induced during aggregation by cAMP (18).

This region forms the pattern of C—X₂—C—X₉—C—X₃—H—X₃—C—X—C; and the cysteines and histidine are conserved in a similar space in 4-1BB, sina, and DG17 proteins. Ten of 24 amino acids between the 4-1BB and sina proteins are identical, and 3 of 24, are conservative substitutes. The conserved pattern suggests that these amino acids are functionally important. The sina protein is localized in the nucleus, suggesting that it has a regulatory function in cells. The fact that the amino acid sequence of 4-1BB contains features like a zinc finger motif, a nuclear protein, and a receptor domain suggests that 4-1BB may play diverse roles during cellular proliferation and differentiation.

4-1BB may represent another cell-surface molecule involved in T cell-APC interactions. The 4-1BB-AP fusion protein specifically bound to mature B-cell lines, anti-p-activated primary B cells, and mature macrophage-cell lines. 4-1BB-AP bound at low or insignificant levels to immature B- and macrophage-cell lines, T-cell clones, T-cell lines, primary culture T cells, and various nonlymphoid-cell lines. Since 4-1BB-AP binds to mature B cells and macrophages, it is possible that signals delivered upon 4-1BB binding may modulate APC functions in some way. This possibility remains to be explored.

Chalupny and colleagues (19) have proposed that 4-1BB Rg, a fusion protein consisting of the extracellular domain of 4-1BB and the Fc region of human IgG, bound to the extracellular matrix (ECM). The highest level of 4-1BB Rg binding was to human vitronectin. In data not shown, an ELISA was performed using 4-1BB-AP and human vitronectin (Yelios Pharmaceuticals/GIBCO-BRL, Grand Island, N.Y.) immobilized at 0.007 μg-10 μg per well on microtiter plates. No binding of 4-1BB-AP based on AP activity was observed. To rule out the possibility that 4-1BB-AP was binding to proteins extrinsically attached to the cell surface (possible extracellular matrix components), B-cell lymphomas were washed in acid conditions prior to the binding assay. 4-1BB-AP still bound specifically to mature B-cell lymphomas. It is still to be determined whether a 4-1BB-ligand specifically expressed on B cells and macrophages exists, and whether 4-1BB-AP may bind to the ECM under particular binding conditions. It is possible that the ECM could facilitate the binding of 4-1BB to a specific cell-surface ligand. B cells and helper T cells interact with each other through receptors on B cells binding to their specific counter-receptors on T cells. It is thought that this interaction results in a cascade of biochemical signaling relays between these two cell types (20). As this interaction proceeds, these cells become committed to enter the S phase of the cell cycle. Initial interactions between TCR and CD4 on T cells, and processed antigen-MHC II on B cells, do not result in B cells capable of entering the cell cycle (21). However, studies from in vitro systems suggest that once T-cells are stimulated, they express newly synthesized or modified cell-surface molecules capable of inducing B cells to enter the cell cycle (22, 23). This T-cell function is not antigen-specific or MHC-restricted (24). In addition, soluble factors are not required for the activated Th induction of B-cell activation (25). Once B cells enter the cell cycle, IL-4 induces B cells to progress from G₁ to S phase. The ability of activated T cells or T-cell membranes to promote the entry of B cells into the cell cycle can be blocked by either cycloheximide or cyclosporin A treatment (26, 27). These newly expressed membrane proteins appear to be “lymphokine-like” in their induction characteristics.

4-1BB has expression properties which meet the requirements of a B-cell costimulator. 4-1BB is inducible by anti-CD3 or TCR-mediated T-cell stimulation, and its expression is sensitive to cyclosporin A as well as cycloheximide treatment (28). Interestingly, paraformaldehyde-fixed SF21-4-1BB cells, synergized with anti-μ in inducing B-cell proliferation. The costimulation of splenic B cells by SF21-4-1BB occurred at optimal (10 μg/ml) and suboptimal (1.0-0.1 μg/ml) doses of anti-μ. The addition of SF21-4-1BB cells to resting B cells, did not result in significant B-cell proliferation. SF21-4-1BB cells did not synergize with TPA or ionomycin, or suboptimal concentrations of LPS in inducing B-cell proliferation.

Although the baculovirus system has been used to express large amounts of recombinant soluble proteins, this system may be utilized for the expression of recombinant cell-surface proteins. The baculovirus infection provides a convenient means to express uniformity high levels of recombinant protein on a per cell basis. It is noteworthy, that the addition of SF21 cells alone did not result in significant levels of costimulation. This can be a potential problem when using cos- or L-cell lines which can exhibit strong costimulator activity on their own.

Another member of the NGFR superfamily, CD40, is expressed on B cells and interacts with gp39, a molecule expressed on activated T cells. The cDNAs encoding the murine (29) and human (30) gp39 proteins have been cloned; this cell surface molecule is a type II membrane protein with homology to tumor necrosis factor. Noelle et al. (31) found that a CD40-immunoglobulin fusion protein, is capable of blocking T cell-induced B-cell proliferation and differentiation in a dose-dependent manner. Armitage et al. have isolated a cDNA for murine gp39 and showed that gp39 could induce B-cell proliferation in the absence of co-stimuli, and result in IgE production in the presence of IL-4-. Hollenbaugh et al. (32) have shown that COS cells transfected with human gp 39 can synergize with either TPA or anti-CD20 in inducing human B-cell proliferation and is able to stimulate B cells without a costimulator only at low levels. These data indicate that CD40 may be one of the B-cell-surface molecules that transmit signals during physical contact with T cells.

Cell-surface receptors communicate with their external milieu by interacting either with soluble factors or other cell surface molecules expressed on neighboring cells. The role of biochemical signals delivered by cell-cell contact versus those delivered by soluble factors interacting with cell surface receptors is not clear. The NGFR superfamily is unusual for the TNFR I and II as well as the NGFR bind to more than one ligand. The TNFRs I and II both bind to TNF-α and TNF-R (33). The NGFR binds to NGF, brain-derived neurotrophic factor, and neurotrophin-3 (34).

In addition, one ligand may function as both a cell surface and soluble ligand. Recent evidence on the CD4-0 ligand, gp39, suggests that this ligand can exist as a membrane bound as well as a soluble ligand (35). It may be possible that 4-1BB is secreted and interacts with B cells in a soluble form as well as a membrane bound form. A member of the NGFR receptor family, CD27, which is 20-expressed on T cells, is secreted in addition to being expressed on the cell surface (36). It is also possible that more than one 1 ligand (soluble and cell surface) may bind to 4-1BB.

Isolation of the Human Homologue, H4-1BB

In order to isolate the human homologue (H4-1BB) of mouse 4-1BB two sets of polymerase chain reaction (PCR) primers were designed. To design the PCR primers, the amino acid sequence among the members of nerve growth factor receptor (NGFR) superfamily were compared because 4-1BB is a member of the superfamily (37). The amino acid sequences employed were mouse 4-1BB (38), human NGFR (39), human tumor necrosis factor receptors (33), human CD40 (40), and human CD27 (6). The areas of sequence conservation among the NGFR superfamily were chosen.

Forward primer I (H4-1BBFI) spans from amino acids 36 to 41 and forward primer II (HR-1BBFII) spans from amino acids 52 to 58 of the mouse 4-1BB. Reverse primer I (H4-1BBRI) spans from amino acids 116 to 121 and reverse primer II (H4-1BBRII) spans from amino acids 122 to 128 of mouse 4-1BB. The regions used as PCR primers in mouse 4-1BB are indicated if FIG. 1.

The degenative oligonucleotide sequence of each primer is as follows:

| | |
|---|---|
| H4-1BBFI: | 5′ TTC TGT CGI AAA TAT AAT CC 3′ |
| | T C A G C C |
| H4-1BBFII: | 5′ TTC TCI TCI ATT GGI GGI CA 3′ |
| | T G G C |
| | A |
| H4-1BBRI: | 5′ CC IAA IGA ACA IGT TTT ACA 3′ |
| | G CT G C G |
| H4-1BBRII: | 5′ TT TTG ATC ATT AAA IGT ICC 3′ |
| | C G G G |

Peripheral blood lymphocytes from normal healthy individuals were isolated and activated with PMA (10 ng/ml) and ionomycin (1 μM). mRNA from the lymphocytes was isolated. Using reverse transcriptase the human lymphocyte mRNA was converted to single-stranded cDNA. The cDNA was then amplified with Taq polymerase with combination of the primers. The combination of primers was as follows: H4-1BBFI vs H4-1BBRI; H4-1BBFI vs H4-1BBRII; H4-1BBFII vs H4-1BBRI; and H4-1BBFII vs H4-1BBRII.

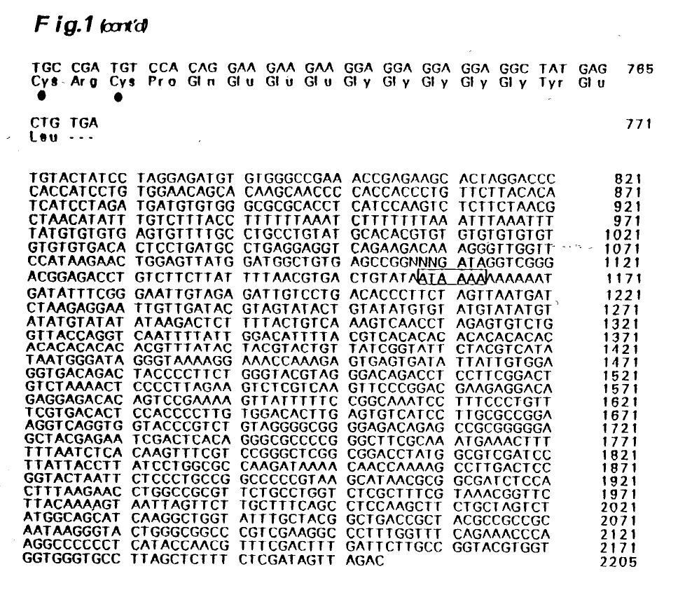

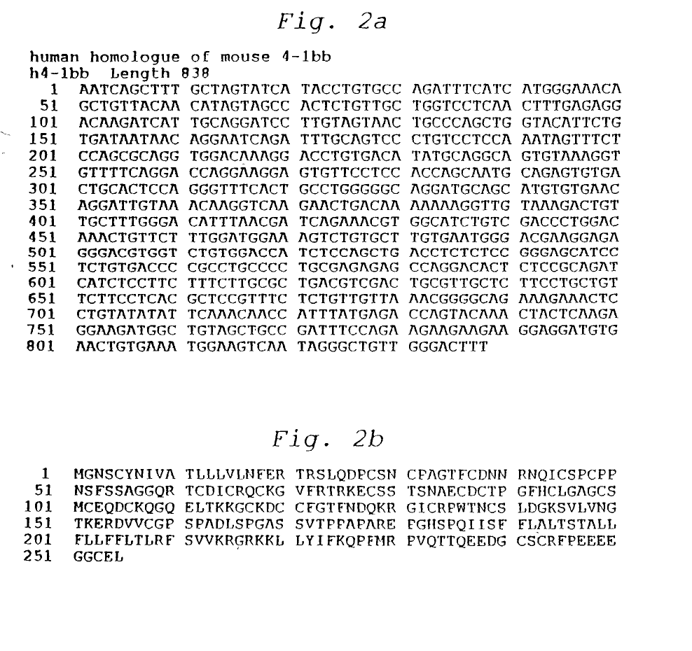

The primer set of H4-1BBFII and H4-1BBRII produced a specific band of ˜240 bp. The 240 bp is an expected size of human 4-1BB if the human homologue protein is similar to mouse 4-1BB in size. The PCR product (240 bp) was cloned in PGEM3 vector and sequenced. One open reading frame of the PCR product was −65% identical to mouse 4-1BB. Therefore, it was concluded that the 240 bp PCR product is the human homologue of mouse 4-1BB. The 240 bp PCR product was used to screen λgt11 cDNA library of activated human T lymphocytes. An ˜0.85 kb cDNA was isolated. The sequence of the cDNA is shown in FIG. 2 and the predicted amino acid sequence is shown in FIG. 2b. The same information is shown is the sequence listing attached to this specification in sequence id. 1.

An expression plasmid to produce H4-1BB-AP fusion protein was constructed. The 5′ portion of the H4-1BB cDNA including sequences encoding the signal sequence and the entire extracellular domain, was amplified by PCR. For correctly oriented cloning, a Hind III site on the 5′ end of the forward primer and a Bg1 II site on the 5′ end of the reverse primer were created.

The Hind III—Bg1 II H4-1BB fragment was inserted into the mammalian expression vector APtaq-1, upstream of the coding sequence for human placental alkaline phosphatase (AP). The oligonucleotides PCR primers used for the amplification of 5′ portion of H4-1BB are as follows:

Forward primer:
5′ AAT AAG CTT TGC TAG TAT CAT ACC T 3′
Reverse primer:
5′ TTA AGA TCT CTG CGG AGA GTG TCC TGG CTC 3′

H4-1BB-AP will be used to identify cells and tissues that express ligand for human 4-1BB (i.e. H4-1BBL). The studies with mouse 4-1BB indicated that the ligand for 4-1BB is on the cell suface. B cells and macrophages were major cells that express 4-1BBL. It is expected that H4-1BBL also expresses on human B cells and macrophages.

A mammalian expression cDNA library will be generated from human cell lines that express H4-1BBL. The library will be screened by [¹²⁵] I-labeled H4-1BB-AP. cDNA for H4-1BBL will then be isolated and characterized. Soluble recombinant H4-1BBL will then be produced. Both H4-1BB-AP and H4-1BBL will be used to suppress or enhance immune responses as described below. Monoclonal antibody to H4-1BB and H4-1BBL will be produced.

According to studies with mouse 4-1BB, 4-1BB acts as a costimulatory signal. It is expected that H4-1BB will act as a costimulatory signal for T cell activation. Mouse 4-1BB helped B cells with proliferation and differentiation. It is expected that H4-1BB will do the same. H4-1BB-AP, H4-1BBL and monoclonal antibody can be used to suppress or enhance human immune responses.

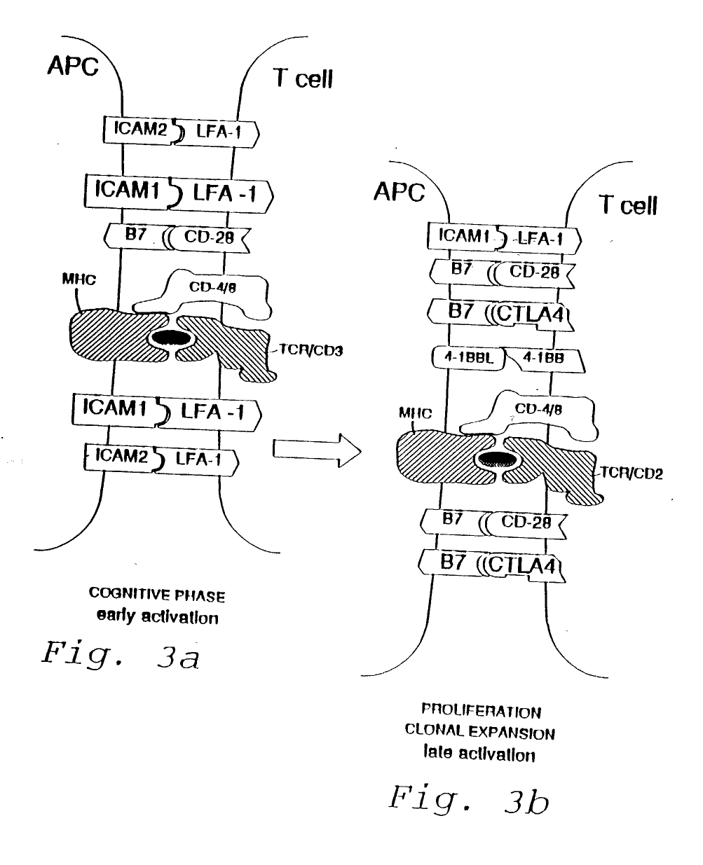

FIGS. 3a and 3b illustrate the molecules involved in T-cell activation. During early T-cell activation (cognitive phase), resting T cells express the TCR/CD3 complex and other “accessory” molecules. Among these constitutively expressed molecules, CD4 (or CD8), LFA-1 and CD28 are probably the ones to receive costimulatory signals. Initial interaction with the TCR/CD3 complex in combination with these ‘accessory’ costimulatory signals leads to subsequent expression of additional receptor molecules such as CD28, CTLA4, and 4-1BB. These newly expressed molecules are probably going to receive additional important costimulatory signals at later stages of T-cell activation (clonal expansion).

Suppression of Immune Responses.

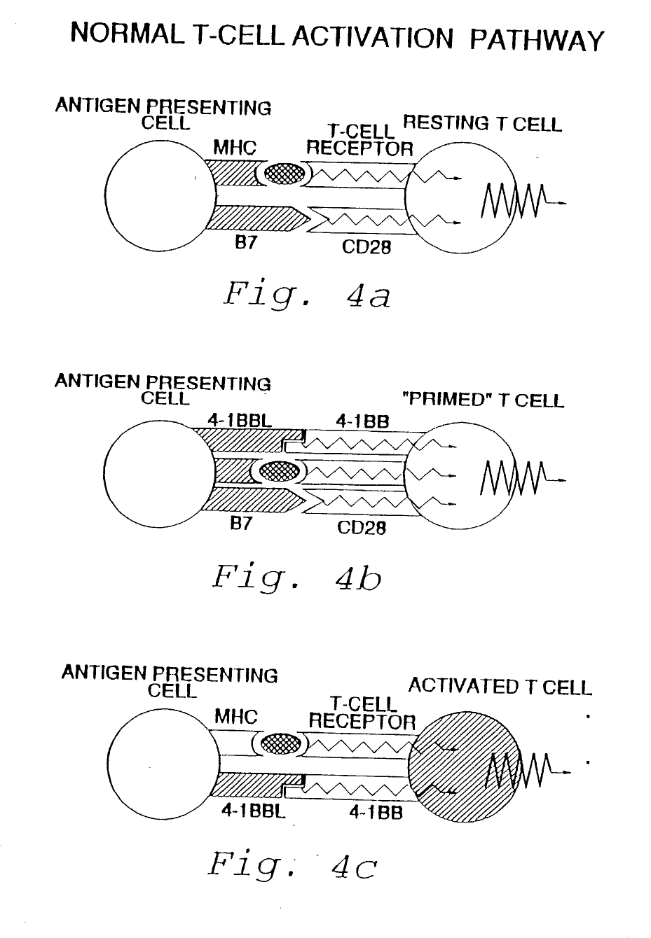

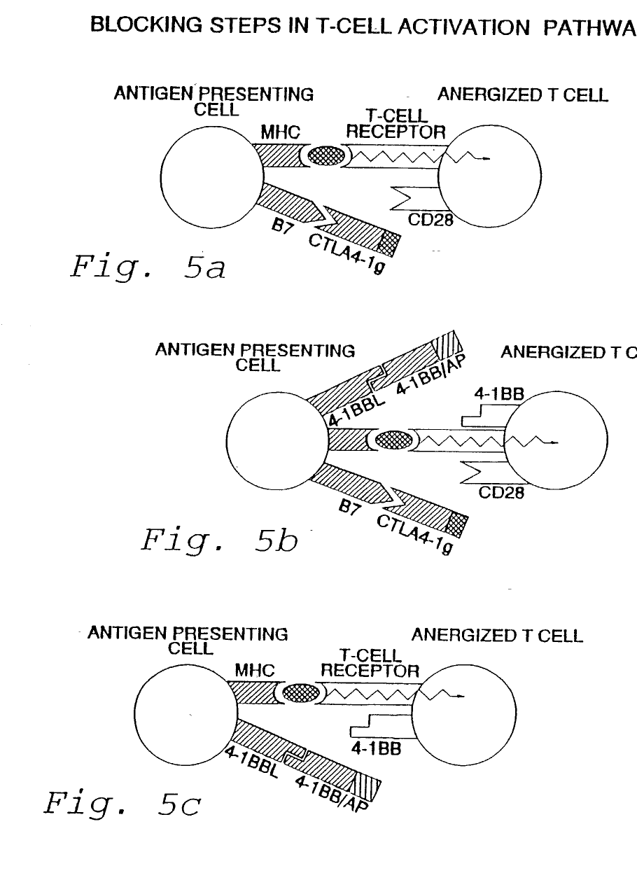

FIGS. 4a-c illustrate a normal T-cell acivation pathway. FIGS. 5a-c illustrate the blocking of immune responses with soluble chimera of 4-1BB. If 4-1BB plays a role in T-cell activation, blocking of the interaction to its ligand on antigen-presenting cells should result in suppression of T-cell dependent immune responses. It is well documented that blocking of the interaction of CD28 to its counter-receptor B7 suppresses in varying degrees, both in vivo antibody production and cell-mediated immune responses. Blocking of both interactions should result in a more effective immunosuppression; since 4-1BB is induced during T-cell activation. Blocking of the interaction of 4-1BB to its ligand may be of importance at later stages of the activation process where the CD28/B7 interaction may no longer be of relevance.

As illustrated with mouse receptor 4-1BB and mouse ligan 4-lBBL above, addition of H4-1BB-AP will coat the H4-1BBL expressing cells and block the normal interaction between H4-1BB and H4-1BBL. This will lead to immunosuppression. This type of immunosuppression is antigen-specific. Therefore it avoids the generalized immunosuppression produced by antiCD3 or cyclosporin A treatments. H4-1BB-AP treatment can be used to treat certain autoimmune diseases and to facilitate organ transplantation.

Immune Enhancement.

H4-1BB may function at the late stage of T cell activation and may be a critical molecule for completion of T cell activation. Most tumors display tumor-specific antigens. One reason, however, why immunogenic tumors can escape host immunity is that tumor-reactive T cells receive inadequate costimulation. The introduction of the costimulatory molecules, such as H4-1BB into the tumor, therefore, could enhance the antitumor immunity of cytotoxic T cells (CTL). H4-1BBL can be expressed in cell-specific fashion. For example, the H4-1BBL can be expressed in melanoma using melanocyte-specific promoter such as tyrosinase promoters. The H4-1BBL-expressing melanoma will stimulate cytotoxic T cells through H4-1BB and activate the melanoma-specific CTL. The activated melanoma-specific CTL can destroy melanoma.

### APPENDIX TO REFERENCES INCORPORATED BY REFERENCE

1. Smith, C. A., Davis, T., Anderson, D., Solam, L., Beckmann, M. P., Jerzy, R., Dower, S. K., Cosman, D., and Goodwin, R. G. 1990. A receptor for tumor necrosis factor defines an unusual family of cellular and viral proteins. Science 248:1019-1023.
2. Ebina, Y., L. Ellis, K. Jaruagin, M. Edery, L. Graf, E. Clauser, J. On, F. Marizrz, Y. W. Kan, J. D. Goldfine, R. A. Roth and W. J. Rutter, 1985, The human insulin receptor cDNA: the structural basis for hormone-activated transmembrane signalling, Cell 40:747.
3. Vassali, R., R. Tedghi, B. Listowska-Bernstein, A. Tartakoff and J. C. Jaton, 1979, Evidence for hydrophobic region within heavy chains of mouse B lymphocyte membrane-bound IgM, Proc. Natl. Acad. Sci. USA 76:5515.
4. Haskins, K., R. Kubo, J. White, M. Pigeon, J. Kappler and P. Marrack, 1983, The major histocompatability complex-restricted antigen receptor on T cells I Isolation with monoclonal antibody, J. Exp. Med. 157:1149.
5. Lesslayer, W. and H. Gmunder, 1986, Biochemical characterization of the 9.3 antigens of human T-cells: stimultaneous expression of disulfide-bonded 90-Kiladalton dimers and free subunits at the cell surface, Mol. Immunol. 23:271.
6. Van Lier, R., J. Borst, T. Vroom, H. Klein, P. Mourik, W. Zeijlemaker and C. Melife, 1987, Tissue distribution and biochemical and functional properties of Tp55 (CD27) a novel T cell differentiation antigen, J. Immunol. 139:1589.
7. Mallett, S., S. Fossum and A. Barclay, 1990, Characterization of the MRC OX40 antigen of activated CD4 positive T lymphocytes-a molecule related to nerve growth factor receptor, EMBO J. 9:1603.
8. Banchereau, J., P. Paoli, A., Valle, E. Garcia and F. Roussel, 1991, Long-term human B cell lines dependent on interleukin-4 and antibody to CD40, Science 251:70.
9. Moeller, D. L., M. K. Jenkins and R. H. Schwartz, 1989, Clonal expansion versus functional colonal inactivation: a co-stimulatory signalling pathway determines the outcome of T cell antigen receptor occupancy, Ann. Rev. Immunol. 7:445.
10. June, D. H., J. A. Ledbetter, P. S. Linsley and C. B. Thompson, 1989, Role of CD28 receptor in T cell activation, Immunol. Today 11:211.
11. Yang, L., B. Jones, A. Aruffo, K. M. Sullivan, P. S. Linsley and C. A. Janeeway, Jr., 1992, Heat stable antigen is a co-stimulatory molecule for CD4 T cell growth, J. Exp. Med. 175:437.
12. Yamori, T., 1992, Molecular mechanisms for generation of neural diversity and specificity: foles of polypeptide factors in development of post-mitotic neurons, Neurosic. Res. 12:545.
13. Liu, Y. J., D. E. Joshua, G. T. Williams, C. A. Smith, J. Gordon and I. C. M. MacLennan, 1989, Mechanism of antigen-driven selection in germinal centres, Nature, 342:929.
14. Jabara, H. H., s. M. Fu, R. S. Geha and D. Vercelli, 1990, CD40 and IfE: synergism between anti-CD40 momoclonal antibody and interleukin 4 in the induction of IgE synthesis by highly purified human B cells, J. Exp. Med. 172:1861.
15. Defrance, R., B. Vanbervliet, F. Briere, I. Durnad, F. Roussle and J. Banchereau, 1992, Interleukin 10 and transforming growth factor β cooperate to induce anti-CD40 activated naive human B cells to secrete immunoglobulin A, J. Exp. Med. 175:671.
16. Donahue, T., Cigan, A., Pahich, E. and Valavicius, B., Mutations at a Zn(II) finger motif in the yeast elF-2 β gene alter ribosomal start-site selection during the scanning process, Cell 54 (1988) 621-632).
17. Carthew, R. W and Rubin, G. M., seven in absentia, a gene required for specification of R7 cell rate in the Drosophila eye, Cell, 63 (1990) 561-577.
18. Driscoll, D. M. and Williams, J. G., Two divergently transcribed genes of Dictyostelium discoideum are cyclic AMP-inducible and coregulated during development, Mol. and Cell. Biol. 7 (1987) 4482-4489.
19. Chalupny, N. J., Peach, R., Hollenbaugh, D., Ledbetter,—J. A., Farr, A. G. and Aruffo, A., 1992, Proc. Natl. Acad. Sci USA 89:10360-10364.
20. Noelle, R. J., and Snow, E. C., 1991, The FASEB J. 5:2770-2776.
21. Noelle, R. and Snow, E., 1990, Immunol. Today 11:361-368.
22. Zurawski, G., Benedik, M., Kamb, B. J., Abrams, J. S., Zurawaki, S. M. and Lee, F. D. (1986) Science 232.772-775.
23. Kinachi, T. (1986) Nature 325,70-73.
24. Gershenfeld, H. K. and Weissman, I. L. Science (1986) 232.854-858.
25. Biggin, M., Gison, T. and Hung, G. (1983 Proc. Natl. Acad. Sci. U.S. Pat. No. 80,3963-3965.
26. Hodgkin, P. D., Yamashita, L. C., Coffman, R. L. and Kehry, M. R., 1990, J. Immunol. 145:2025-2034.
27. Barlett, W. C., McCann, J., Shephaer, D. M., Roy, M. and Noelle, R. J., 1990, J. Immunol. 145:3956-3962.
28. Kwon, B. S., Kestler, D. P., Eshhar, Z., Oh, K., and Wakulchik, M. 1989. Expression characteristics of two potential T cell mediator genes. Cell. Immunol. 121:414-422.
29. Armitage, R., Fanslow, W., Strockbine, L., Sato, T., Clifford, K., MacDuff, B., Anderson, D., Gimpel, S., Davis-Smith, T., Maliszewski, C., Clark, E., Smith, C., Grabstein, K., Cosman, D. and Spriggs, M., 191, Nature 357:80-82.
30. Kwon, B., Kestler, D., Lee, E., Wakulchik, M. and Young J. (1988) J. Exp. Med) (1988) (In press).
31. Noelle, R. J., Roy, M., Shepherd, D. M., Stamenkovic, I., Ledbetter, J. A. and Aruffo, A., 1992, Proc. Natl. Acad. Sci. USA 89:6550-6554.
32. Hollenbaugh, D., Grosmaier, L. S., Kullas, C. D., Chalupny, N. J., Braesch-Andersen, S., Noelle, R. J., Stamenkovic, I., Ledbetter, J. A. and Aruffo, A., 1992, EMBO 11:4314-4321.
33. Schall, T. J., M. Lewis, K. J. Koller, A. Lee, G. C. Rice, G. H. W. Wong, T. Gatanaga, G. A. Granger, R. Lentz, H. Raab, W. J. Kohr and D. V. Goeddel, 1990, Molecular cloning and ex8pression of a receptor for human tumor necrosis factor, Cell 61:361.
34. Klein, R., Nanduri, V., Jing, S., Lamballe, F., Tapley, P., Bryant, S., Cordon-Cardo, C., Jones, K. R., Reichardt, L. F. and Barbacid, M., 1991, Cell 66:395-403.
35. Armitage, R. J., Sato, T. A., Macduff, B. M., Clifford, K. N., Alpert, A. R., Smith, C. A. and Fanslow, W. C., 1992, Eu8r. J. Immunol. 22:2071-2076.
36. Hintzen, R. Q., deJong, R., Hack, E. E., Chamuleau, M., de Vries, E. F. R., ten Berge, I. J. M., Borst, J. and van Lier, R. A. W., 1991, J. Immunol. 147:29-35.
37. Mallett, S., and Barclay, A. N. 1991. A new super-family of cell surface proteins related to the nerve growth factor receptor. Immunol. Today. 12:220-223.
38. Kwon, B. S., and Weissman, S. M. 1989. cDNA sequences of two inducible T-cell genes. Proc. Natl. Acad. Sci. USA. 86:1963-1967.
39. Johnson, D., Lanahan, A., Buck C. R., Sehgal, A., Morgan, C., Mercer, E., Bothwell, M., and Chao, M. 1986. Expression and structure of the human NGF receptor. Cell 47:545-554.
40. Stamenkovic, I., Clark, E., and Seed, B. 1989. A B-lymphocyte activation molecule related to the nerve growth factor receptor and induced by cytokines in carcinomas. EMBO. J. 8:1403-1408.

The foregoing description has been directed to particular embodiments of the invention in accordance with the requirements of the Patent Statutes for the purposes of illustration and explanation. It will be apparent, however, to those skilled in this art that many modifications and changes will be possible without departure from the scope and spirit of the invention. It is intended that the following claims be interpreted to embrace all such modifications.

I claim:

1. A cDNA encoding for human receptor protein H4-1BB.

2. The cDNA of claim 1 having a nucleotide sequence as shown in FIG. 2.

3. The cDNA of claim 1, identified as pH 4-1BB deposited at the Agricultural Research Service Culture Collection with the accession number NRRL B21131.

4. The cDNA of claim 2 and fragments and derivatives thereof, wherein said fragments and derivatives can be used as a probe to isolate DNA sequences encoding for proteins similar to the receptor protein encoded by said cDNA.

5. The receptor protein H4-1BB produced by
a) inserting the cDNA of H4-1BB into an appropriate expression vector,
b) transfecting said expression vector into an appropriate transfection host,
c) growing said transfected hosts in appropriate culture media and
d) purifying the receptor protein from said culture media.

6. A protein having the amino acid sequence shown in FIG. 2.

7. The protein of claim 6 and fragments and derivatives thereof, wherein said fragments and derivatives:
a) can be used as a probe to identify ligands to receptor protein H4-1BB;
b) can be used to stimulate proliferation B-cell's expressing H4-1BB ligands; or
c) can be used to block H4-1BB ligand binding.

8. A monoclonal antibody against H4-1BB which specifically recognizes receptor protein H4-1BB.

9. A hybridoma capable of producing a monoclonal antibody against H4-1BB which specifically recognizes receptor protein H4-1BB.

10. The method of using the monoclonal antibody of claim 8 to enhance T-cell proliferation comprising the step of treating T-cells that have expressed receptor protein H4-1BB with said monoclonal antibody.

11. The method of claim 12 further comprising the step of conducting said treatment in the presence of protein tyrosinase kinase.

12. The method of using the monoclonal antibody of claim 8 to enhance T-cell activation comprising the step of treating T-cells that have expressed receptor protein H4-1BB with said monoclonal antibody.

13. The method of claim 12 further comprising the step of conducting said treatment in the presence of protein tyrosinase kinase.

14. A fusion protein for detecting cell membrane ligands to human receptor protein H4-1BB, comprising:
a) at least a portion of said receptor protein H4-1BB corresponding to the extracellular portion of said receptor protein H4-1BB such that said portion of said receptor protein H4-1BB binds to said cell membrane ligands; and
b) a detection protein bound to said portion of said receptor protein H4-1BB such that ligand binding can be detected by relative activity assays for said detection protein.

15. The fusion protein of claim 14 wherein said detection protein is alkaline phosphatase.

16. A method of detecting cell membrane ligands to human receptor protein H4-1BB, comprising:
a) providing a fusion protein including:
1) at least a portion of said receptor protein H4-1BB corresponding to the extracellular portion of said receptor protein H4-1BB such that said portion of said receptor protein H4-1BB binds to said cell membrane ligands, and
2) a detection protein bound to said portion of said receptor protein H4-1BB such that ligand binding can be detected by relative activity assays for said detection protein;
b) placing said fusion protein in the presence of a cell suspected to express said receptor protein H4-1BB;
c) washing said cell of any fusion protein not bound to said cell membrane ligands;
d) placing said washed cells in the presence of a substrate for said detection protein and measuring the relative activity of said detection protein.

17. The method of claim 16 wherein said detection protein is alkaline phosphatase.

18. A method of inducing B-cell proliferation comprising the step of treating B-cells that have expressed a ligand to human receptor protein H4-1BB with cells that have expressed receptor protein H4-1BB.


SEQUENCE LISTING (8 sequences)

SEQ ID NO: 1 - DNA (cDNA to mRNA), 838 bp, Homo sapiens; double-stranded, linear
  CDS 41..805: /codon_start= 41 /product= “H4-1BB” /number= 1
  mat_peptide 41..802: /codon_start= 41 /product= “H4-1BB” /number= 1
  hypothetical: NO
  anti-sense: NO
  original source individual isolate: H4-1BB #1
  original source developmental stage: Differentiated T-cell
  original source cell type: Lymphocyte
aatcagctttgctagtatcatacctgtgccagatttcatcatgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagctgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtgaaatggaagtcaatagggctgttgggacttt

SEQ ID NO: 2 - protein, 255 aa
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 3 - DNA (other nucleic acid), 20 bp; single-stranded, linear
  description: /desc = “PCR Primer”
  hypothetical: NO
  anti-sense: NO
ttytgymgaaartayaaycc

SEQ ID NO: 4 - DNA (other nucleic acid), 20 bp; single-stranded, linear
  description: /desc = “PCR Primer”
  hypothetical: NO
  anti-sense: NO
ttytcstscahtggtggaca

SEQ ID NO: 5 - DNA (other nucleic acid), 20 bp; single-stranded, linear
  description: /desc = “PCR Primer”
  hypothetical: NO
  anti-sense: NO
cccargswrcaggtyttrca

SEQ ID NO: 6 - DNA (other nucleic acid), 20 bp; single-stranded, linear
  description: /desc = “PCR Primer”
  hypothetical: NO
  anti-sense: NO
ttytgrtcrttraatgttcc

SEQ ID NO: 7 - DNA (other nucleic acid), 25 bp; single-stranded, linear
  description: /desc = “PCR Primer”
  hypothetical: NO
  anti-sense: NO
aataagctttgctagtatcatacct

SEQ ID NO: 8 - DNA (other nucleic acid), 30 bp; single-stranded, linear
  description: /desc = “PCR Primer”
  hypothetical: NO
  anti-sense: NO
ttaagatctctgcggagagtgtcctggctc